(19) United States
(12) Patent Application Publication
(10) Pub. No.: US 2008/0114218 A1
(43) Pub. Date: May 15, 2008

(54) CONDITION IMPROVEMENT ADVISOR

(75) Inventors: Mari Suyama, Aichi (JP); Toshinobu Mizutani, Aichi (JP); Masato Nishikawa, Aichi (JP)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: KABUSHIKI KAISHA TOKAI RIKA DENKI SEISAKUSHO, Aichi (JP)

(21) Appl. No.: 11/936,985

(22) Filed: Nov. 8, 2007

(30) Foreign Application Priority Data
Nov. 10, 2006 (JP) 2006-305461

(51) Int. Cl. A61B 5/00

(52) U.S. Cl. 600/300

(57) ABSTRACT

A condition improvement advisor for monitoring a condition related to the user and improving the monitored condition. The condition improvement advisor includes a sensor unit for detecting the monitored condition and outputting data representing the user's present state when carried by the user. A present state determination circuit determines from the data representing the user's present state whether or not the user's present state should be improved. When determining that the user's present state should be improved, the present state determination circuit generates advice indicating an item that would improve the present state. An output device outputs the advice generated by the present state determination circuit in a form understandable to a human.

### BACKGROUND OF THE INVENTION

The present invention relates to a condition improvement advisor and a main unit for a condition improvement advisor.

Japanese Laid-Open Patent Publication No. 2005-34520 describes a physical state monitoring system including a perspiration sensor arranged in a portable device carried by a driver. The portable device transmits data associated with the driver's perspiration amount, which is detected by the sensor. The data is received by an automobile. The automobile determines the physical state of the driver obtained from the data and shows the physical state on a display unit. A heart beat (pulse) sensor or a blood pressure sensor may be arranged in the portable device in lieu of or in addition to the perspiration sensor. In such a case, the driver's physical state is determined from the driver's heart beat (pulse) or blood pressure in lieu of or in addition to the driver's perspiration amount and shown on the display.

### SUMMARY OF THE INVENTION

The physical state monitoring system of the above publication provides for visualization of the physical state of a driver based on sensor detection data. However, when the physical state of the driver deteriorates, the system does not issue any warnings and does not advise on what the driver should do in such a case.

One aspect of the present invention is a condition improvement advisor for monitoring a condition related to a user carrying the condition improvement advisor and improving the monitored condition. The condition improvement advisor includes a sensor unit for detecting the monitored condition and outputting data representing the user's present state when carried by the user. A present state determination circuit determines from the data representing the user's present state whether or not the user's present state should be improved. When determining that the user's present state should be improved, the present state determination circuit generates advice indicating an item that would improve the present state. An output device outputs the advice generated by the present state determination circuit in a form understandable to a human.

Other aspects and advantages of the present invention will become apparent from the following description, taken in conjunction with the accompanying drawings, illustrating by way of example the principles of the invention.

### BRIEF DESCRIPTION OF THE DRAWINGS

The invention, together with objects and advantages thereof, may best be understood by reference to the following description of the presently preferred embodiments together with the accompanying drawings in which:

FIG. 1(a) is a plan view of a condition improvement advisor according to a preferred embodiment of the present invention;

FIG. 1(b) is a partially enlarged cross-sectional view of the condition improvement advisor of FIG. 1(a); and

FIG. 2 is a block diagram of the condition improvement advisor of FIG. 1(a).

### DETAILED DESCRIPTION OF THE PREFERRED EMBODIMENTS

A condition improvement advisor according to a preferred embodiment of the present invention will now be described. In the preferred embodiment, the condition improvement advisor monitors conditions including the environmental state and physical state of a user and issues advices for improving the monitored condition.

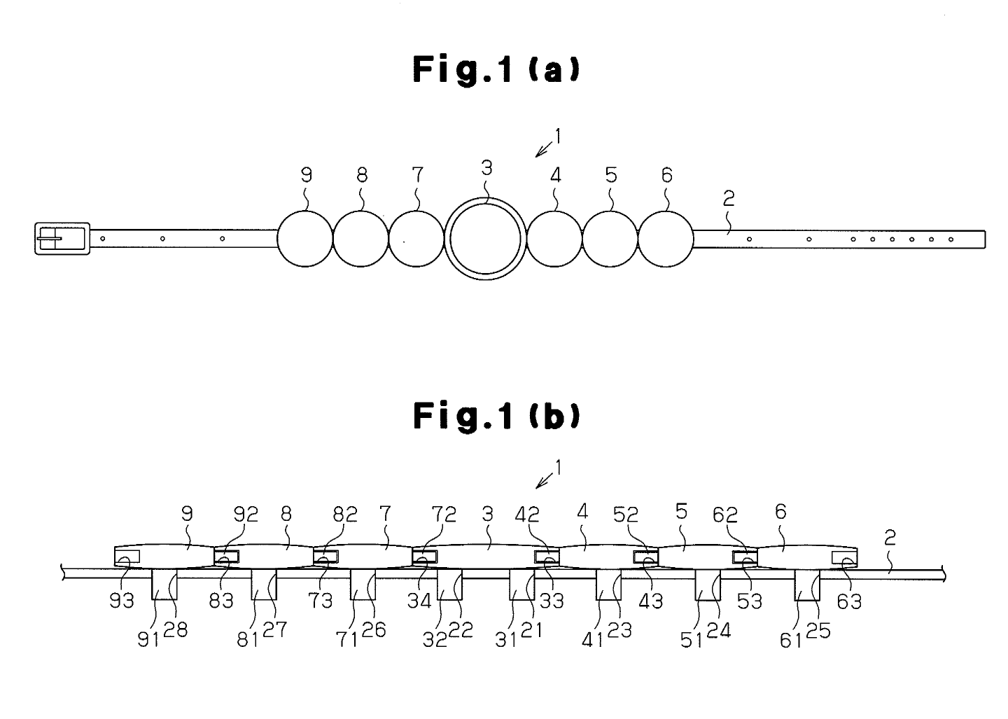

As shown in FIGS. 1(a) and 1(b), the condition improvement advisor 1 includes a main unit 3 attached to a band 2, which functions as a supporting member. An ambient temperature sensor unit 4, a humidity sensor unit 5, and an ultraviolet sensor unit 6 are connected in series along the band 2 in this order at one side of the main unit 3. A body temperature sensor unit 7, a heart beat sensor unit 8, and a skin moisture sensor unit 9 are connected in series along the band 2 in this order at the other side of the main unit 3. The user puts on the condition improvement advisor 1 by wearing the band 2 on his or her arm. The condition improvement advisor 1 is also referred to as a portable remote condition improvement advisor or a wearable condition improvement advisor.

The ambient temperature sensor unit 4, the humidity sensor unit 5, the ultraviolet sensor unit 6, the body temperature sensor unit 7, the heart beat sensor unit 8, and the skin moisture sensor unit 9 are each smaller than the main unit 3. In the preferred embodiment, the sensor units 4 to 9 are circular and have identical shapes and dimensions. Each sensor unit 4 to 9 has a thickness that is substantially the same as that of the main unit 3. Each sensor unit 4 to 9 is one example of an external sensor unit.

The band 2 has flexibility and is elastically deformable. Two attachment holes 21 and 22 extend through the longitudinally middle part of the band 2. The attachment hole 21 is formed slightly towards a distal end of the band 2 from a longitudinally central point of the band 2. The attachment hole 22 is formed slightly towards a basal end of the band 2 from the longitudinally central point of the band 2. Two pins 31 and 32 are formed at a central part of the rear surface of the main unit 3. The main unit 3 is detachably attached to the band 2 by fitting the pin 31 into the attachment hole 21 and fitting the pin 32 into the attachment hole 22.

The pins 31 and 32 each have a diameter that is slightly larger than that of the attachment holes 21 and 22. The pins 31 and 32 are press-fitted to the attachment holes 21 and 22 against the elastic force of the band 2. The pins 31 and 32 each have a length that is greater than the depth of the attachment holes 21 and 22, that is, the thickness of the band 2. The pins 31 and 32 each have a distal end face that comes into close contact with a user's arm when the band 2 is worn. The projecting length of the pins 31 and 32 from the rear surface of the band 2 is set so that the user does not feel pain at the arm when wearing the band 2. The distal end face of each of the pins 31 and 32 may be rounded so that the user does not feel pain at the arm.

The distal end face of each of the pins 31 and 32 defines a surface that contacts the user's arm. A reference potential electrode and a communication electrode are exposed from the distal end face of the pin 31 and the distal end face of the pin 32. The reference potential electrode and the communication electrode use the user's body as a signal transmission path so as to realize in-body communication between the condition improvement advisor 1 and the automobile. The reference potential electrode, the communication electrode, and the in-body communication are not essential parts of the present embodiment and thus will not be described in detail.

A line of spaced attachment holes 23 to 25 are formed towards the distal end of the band 2 from the attachment hole 21. A line of spaced attachment holes 26 to 28 are formed towards the basal end of the band 2 from the attachment hole 22. The attachment holes 23 to 28 have the same diameter.

A pin 41 is formed at the middle part of the rear surface of the ambient temperature sensor unit 4. The ambient temperature sensor unit 4 is detachably attached to the band 2 by fitting the pin 41 into the attachment hole 23.

The pin 41 has a diameter that is slightly greater than that of the attachment hole 23. The pin 41 is press-fitted to the attachment hole 23 against the elastic force of the band 2. The pin 41 has a length that is greater than the depth of the attachment hole 23, that is, the thickness of the band 2. The pin 41 has a distal end face that comes into close contact with the user's arm of the user when the band 2 is worn. The projecting length of the pin 41 from the rear surface of the band 2 is set so that the user does not feel pain at the arm when the band 2 is worn. The distal end face of the pin 41 may be rounded so that the user does not feel pain at the arm.

In the ambient temperature sensor unit 4, an ambient temperature sensor is exposed from a front surface, which is opposite the rear surface of the pin 41. The temperature sensor detects the ambient temperature (also referred to as environmental temperature) at the location of the user. The ambient temperature is one parameter of the user's environmental state.

A pin 51 is formed at a middle part of the rear surface of the humidity sensor unit 5. The humidity sensor unit 5 is detachably attached to the band 2 by fitting the pin 51 into the attachment hole 24.

The pin 51 has a diameter that is slightly larger than that of the attachment hole 24. The pin 51 is press-fitted to the attachment hole 24 against the elastic force of the band 2. The pin 51 has a length that is greater than the depth of the attachment hole 24, that is, the thickness of the band 2. The pin 51 has a distal end face that comes into close contact with the user's arm when the band 2 is worn. The projecting length of the pin 51 from the rear surface of the band 2 is set so that the user does not feel pain at the arm when the band 2 is worn. The distal end face of the pin 51 may be rounded so that the user does not feel pain at the arm.

In the humidity sensor unit 5, a humidity sensor is exposed from a front surface, which is opposite the rear surface of the pin 51. The humidity sensor detects the humidity at the location of the user. The humidity is one parameter of the user's environmental state.

A pin 61 is formed at a middle part of the rear surface of the ultraviolet sensor unit 6. The ultraviolet sensor unit 6 is detachably attached to the band 2 by fitting the pin 61 into the attachment hole 25.

The pin 61 has a diameter that is slightly larger than that of the attachment hole 25. The pin 61 is press-fitted to the attachment hole 25 against the elastic force of the band 2. The pin 61 has a length that is greater than the depth of the attachment hole 25, that is, the thickness of the band 2. The pin 61 has a distal end face that comes into close contact with the user's arm when the band 2 is worn. The projecting length of the pin 61 from the rear surface of the band 2 is set so that the user does not feel pain at the arm when the band 2 is worn. The distal end face of the pin 61 may be rounded so that the user does not feel pain at the arm.

In the ultraviolet sensor unit 6, an ultraviolet sensor is exposed from a front surface, which is opposite the rear surface of the pin 61. The ultraviolet sensor detects the intensity or dose of ultraviolet light received by the user. The intensity or dose of ultraviolet light is one parameter of the user's environmental state.

A pin 71 is formed at the middle part of the rear surface of the body temperature sensor unit 7. The body temperature sensor unit 7 is detachably attached to the band 2 by fitting the pin 71 into the attachment hole 26.

The pin 71 has a diameter that is slightly greater than that of the attachment hole 26. The pin 71 is press-fitted to the attachment hole 26 against the elastic force of the band 2. The pin 71 has a length that is greater than the depth of the attachment hole 26, that is, the thickness of the band 2. The pin 71 has a distal end face that comes into close contact with the user's arm when the band 2 is worn. The projecting length of the pin 71 from the rear surface of the band 2 is set so that the user does not feel pain at the arm when the band 2 is worn. The distal end face of the pin 71 may be rounded so that the user does not feel pain at the arm.

The distal end face of the pin 71 defines a surface that contacts the user's arm. A body temperature sensor is exposed from the distal end face of the pin 71. The body temperature sensor detects the user's skin temperature. The skin temperature is one parameter of the user's physical state.

A pin 81 is formed at the middle part of the rear surface of the heart beat sensor unit 8. The heart beat sensor unit 8 is detachably attached to the band 2 by fitting the pin 81 into the attachment hole 27.

The pin 81 has a diameter that is slightly larger than that of the attachment hole 27. The pin 81 is press-fitted to the attachment hole 27 against the elastic force of the band 2. The pin 81 has a length that is greater than the depth of the attachment hole 27, that is, the thickness of the band 2. The pin 81 has a distal end face that comes into close contact with the user's arm when the band 2 is worn. The projecting length of the pin 81 from the rear surface of the band 2 is set so that the user does not feel pain at the arm when the band 2 is worn. The distal end face of the pin 81 may be rounded so that the user does not feel pain at the arm.

The distal end face of the pin 81 defines a surface that contacts the user's arm. A heart beat sensor is exposed from the distal end face of the pin 81. The heart beat sensor detects the heart beat of the user. The heart beat is one parameter of the user's physical state.

A pin 91 is formed at the middle part of the rear surface of the skin moisture sensor unit 9. The skin moisture sensor unit 9 is detachably attached to the band 2 by fitting the pin 91 into the attachment hole 28.

The pin 91 has a diameter that is slightly larger than that of the attachment hole 28. The pin 91 is press-fitted to the attachment hole 28 against the elastic force of the band 2. The pin 91 has a length that is greater than the depth of the attachment hole 28, that is, the thickness of the band 2. The pin 91 has a distal end face that comes into close contact with the user's arm when the band 2 is worn. The projecting length of the pin 91 from the rear surface of the band 2 is set so that the user does not feel pain at the arm when the band 2 is worn. The distal end face of the pin 91 may be rounded so that the user does not feel pain at the arm.

The distal end face of the pin 91 defines a surface that contacts the user's arm. A skin moisture sensor is exposed from the distal end face of the pin 91. The skin moisture sensor detects the user's skin moisture content. The skin moisture content is one parameter of the user's physical state.

A first connection structure is formed in the side of the main unit 3. The first connection structure is formed by a recess such as a female connector 33. A second connection structure is formed opposite to the female connector 33 in the side of the main unit 3. The second connection structure is formed by a recess such as a female connector 34.

A first connector that mates with the female connector 33 (first connection structure) is formed in the side of the ambient temperature sensor unit 4, which serves as a first sensor unit. The first connector is formed by a projection such as a male connector 42. The ambient temperature sensor unit 4 is detachably attached to the main unit 3 by fitting the male connector 42 to the female connector 33. A second connector is formed opposite to the male connector 42 in the side of the ambient temperature sensor unit 4. The second connector is formed by a recess such as a female connector 43.

A third connector that mates with the female connector 43 (second connector) is formed in the side of the humidity sensor unit 5, which serves as a second sensor unit. The third connector is formed by a projection such as a male connector 52. The humidity sensor unit 5 is detachably attached to the ambient temperature sensor unit 4 by fitting the male connector 52 to the female connector 43. A fourth connector is formed opposite to the male connector 52 in the side of the humidity sensor unit 5. The fourth connector is formed by a recess such as a female connector 53.

A male connector 62 that mates with the female connector 53 (fourth connector) is formed in the side of the ultraviolet sensor unit 6, which serves as a third sensor unit. The ultraviolet sensor unit 6 is detachably attached to the humidity sensor unit 5 by fitting the male connector 62 to the female connector 53. A female connector 63 is formed opposite to the male connector 62 in the side of the ultraviolet sensor unit 6. The female connector 63 does not receive any male connector in the present embodiment.

A fifth connector that mates with the female connector 34 (second connection structure) is formed in the side of the body temperature sensor unit 7, which serves as a fourth sensor unit. The fifth connector is formed by a projection such as a male connector 72. The body temperature sensor unit 7 is detachably attached to the main unit 3 by fitting the male connector 72 to the female connector 34. A sixth connector is formed opposite to the male connector 72 in the side of the body temperature sensor unit 7. The sixth connector is formed by a recess such as a female connector 73.

A seventh connector that mates with the female connector 73 (sixth connector) is formed in the side of the heart beat sensor unit 8, which serves as a fifth sensor unit. The seventh connector is formed by a projection such as a male connector 82. The heart beat sensor unit 8 is detachably attached to the body temperature sensor unit 7 by fitting the male connector 82 to the female connector 73. An eighth connector is formed opposite to the male connector 82 in the side of the heart beat sensor unit 8. The eighth connector is formed by a recess such as a female connector 83.

A male connector 92 that mates with the female connector 83 (eighth connector) is formed in the side of the skin moisture sensor unit 9, which serves as a sixth sensor unit. The skin moisture sensor unit 9 is detachably attached to the heart beat sensor unit 8 by fitting the male connector 92 to the female connector 83. A female connector 93 is formed opposite to the male connector 92 in the side of the skin moisture sensor unit 9. The female connector 93 does not receive any male connector in the present embodiment.

The female connectors 33, 34, 43, 53, 63, 73, 83, and 93 have the same structure. The male connectors 42, 52, 62, 72, 82, and 92 have the same structure. Therefore, in the present embodiment, except for its own sensor unit female connector, the male connectors 42, 52, 62, 72, 82, and 92 can each be mated with any one of the female connectors 33, 34, 43, 53, 63, 73, 83, and 93.

The female connectors 33, 34, 43, 53, 63, 73, 83, and 93 and male connectors 42, 52, 62, 72, 82, and 92 each have a contact or a terminal (not shown). When one female connector is fitted with one male connector, the contact or the terminal of the female connector is electrically connected to the contact or the terminal of the male connector. The sensor units 4 to 9 thus have connection compatibility.

The electrical configuration of the condition improvement advisor 1 will now be described.

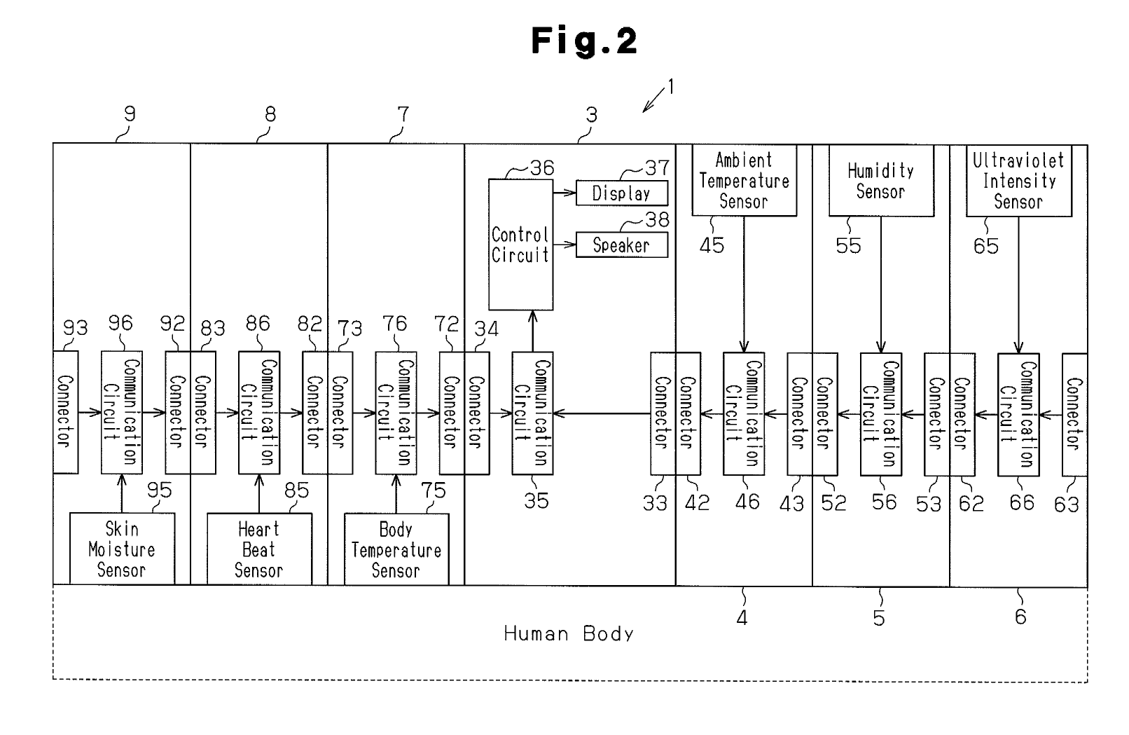

As shown in FIG. 2, the ambient temperature sensor unit 4 is electrically connected to the main unit 3 of the condition improvement advisor 1 by the female connector 33 and the male connector 42. The humidity sensor unit 5 is electrically connected to the ambient temperature sensor unit 4 by the female connector 43 and the male connector 52, and the ultraviolet sensor unit 6 is electrically connected to the humidity sensor unit 5 by the female connector 53 and the male connector 62.

The body temperature sensor unit 7 is electrically connected to the main unit 3 of the condition improvement advisor 1 by the female connector 34 and the male connector 72. The heart beat sensor unit 8 is electrically connected to the body temperature sensor unit 7 by the female connector 73 and the male connector 82, and the skin moisture sensor unit 9 is electrically connected to the heart beat sensor unit 8 by the female connector 83 and the male connector 92.

The ultraviolet sensor unit 6 detects the intensity or dose of ultraviolet light with an ultraviolet intensity or dose sensor 65 exposed from its front surface, converts the data representing the ultraviolet light intensity or dose with a communication circuit 66 into a format easily handled by the main unit 3, and provides the converted data to an adjacent external device (in this case, the humidity sensor unit 5) through the male connector 62. The ultraviolet sensor unit 6 provides the data acquired from a preceding external device (in this case, there is no external device connected to the female connector 63) via the female connector 63 to the following external device (in this case, humidity sensor unit 5) via the communication circuit 66 and the male connector 62.

The humidity sensor unit 5 detects the humidity with a humidity sensor 55 exposed from its front surface, converts the data representing the ambient humidity with a communication circuit 56 into a format easily handled by the main unit 3, and provides the converted data to the adjacent external device (in this case, ambient temperature sensor unit 4) via the male connector 52. The humidity sensor unit 5 provides the data acquired from the preceding external device (in this case, ultraviolet sensor unit 6) via the female connector 53 to the following external device (in this case, ambient temperature sensor unit 4) via the communication circuit 56 and the male connector 52.

The ambient temperature sensor unit 4 detects the ambient temperature with a temperature sensor 45 exposed from its front surface, converts the data representing the ambient temperature with a communication circuit 46 into a format easily handled by the main unit 3, and provides the converted data to the adjacent external device (in this case, main unit 3) via the male connector 42. The ambient temperature sensor unit 4 provides the data acquired from the preceding external device (in this case, humidity sensor unit 5) via the female connector 43 to the following external device (in this case, main unit 3) via the communication circuit 46 and the male connector 42.

In the skin moisture sensor unit 9, a skin moisture sensor 95 detects the user's skin moisture content. A communication circuit 96 converts the data representing the user's skin moisture content to a format easily handled by the main unit 3. Then, the communication circuit 96 provides the converted data to the adjacent external device (in this case, heart beat sensor unit 8) via the male connector 92. When there is a preceding external device connected to the skin moisture sensor unit 9 by the female connector 93 (in the preferred embodiment, there is no external device connected by the female connector 93), the skin moisture sensor unit 9 provides the data acquired from the preceding external device to the following external device (in this case, heart beat sensor unit 8) via the communication circuit 96 and the male connector 92.

In the heart beat sensor unit 8, a heart beat sensor 85 detects the user's heart beat. A communication circuit 86 converts the data representing the user's heart beat into a format easily handled by the main unit 3. Then, the communication circuit 86 provides the converted data to the adjacent external device (in this case, body temperature sensor unit 7) via the male connector 82. The heart beat sensor unit 8 provides the data acquired from the preceding external device (in this case, skin moisture sensor unit 9) via the male connector 83 to the following external device (in this case, body temperature sensor unit 7) via the communication circuit 86 and the male connector 82.

In the body temperature sensor unit 7, a body temperature sensor 75 detects the user's skin temperature. A communication circuit 76 converts the data representing the user's skin temperature into a format easily handled by the main unit 3. Then, the communication circuit 76 provides the converted data to the adjacent external device (in this case, main unit 3) via the male connector 72. The body temperature sensor unit 7 provides the data acquired from the preceding external device (in this case, heart beat sensor unit 8) via the female connector 73 to the following external device (in this case, main unit 3) via the communication circuit 76 and the male connector 72.

The main unit 3 includes a communication circuit 35 that specifies the sensor units associated with the data acquired from the adjacent external device via the female connector 33 (in this case, the ambient temperature sensor unit 4). The communication circuit 35 further specifies the sensor units associated with the data acquired from the adjacent external device via the female connector 34 (in this case, the body temperature sensor unit 7). The communication circuit 35 of the main unit 3 generates data in which the specification result and the level of data are associated and provides the data to a control circuit 36, which functions as a present state determination circuit. The control circuit 36e analyzes the data. When determining that the user's conditions should be improved, the control circuit 36 activates an output device (at least either one of a display 37 and a speaker 38) to issue advice for improving the user's conditions.

The control circuit 36 analyzes the data provided from the sensor units and generates a message in accordance with the analysis result. The method is output through the output device. For example, the control circuit 36 may select and output through the output device a message that is in accordance with the analysis result of the data provided from the sensor units from visual messages and audible messages stored in a message database. The message database may be stored in a storage device such as a memory that is accessible by the control circuit 36. The storage device is, for example, an internal storage device incorporated in the control circuit 36 or an external storage device connected to the control circuit 36.

Examples of advisories that are associated with conditions that should be improved will now be discussed.

In a first example, the user of the condition improvement advisor 1 stops driving a vehicle during the wintertime and walks outdoor from a garage to his or her house.

In this case, the user may be exposed to cold and dry air for a long time such that the user's skin temperature becomes low and the user's skin moisture content decreases. As a result, the skin temperature detected by the body temperature sensor 75 becomes less than a reference skin temperature (enters an abnormal range). Further, the skin moisture content detected by the skin moisture sensor 95 becomes less than a reference skin moisture content (enters an abnormal range). Based on the data provided from the body temperature sensor 75 and the skin moisture sensor 95, the control circuit 36 determines that the user's skin temperature is less than the reference skin temperature and that the user's skin moisture content is less than the reference skin moisture content. Then, the control circuit 36 activates the display 37 and the speaker 38 and issues advice for increasing the user's skin temperature to the reference skin temperature so that the skin temperature enters a normal range. Further, the control circuit 36 issues advice for increasing the user's skin moisture content to the reference skin moisture content so that the skin moisture content enters a normal range. For example, the control circuit 36 shows a text message or a picture message (e.g., icon or dynamic image) on the display 37. The content of the text message or picture message may be “Skin temperature is low and skin is dry. When reaching home, set air conditioner to high temperature and activate humidifier.” Further, the control circuit 36 outputs the content of the message from the speaker 38.

In this manner, in the first example, when determining that the user's conditions should be improved, the control circuit 36 activates the display 37 and/or the speaker 38 and advises the user in a manner understandable to the user on the actions that are necessary to improve the user's present conditions. The advice describes the user's present conditions (e.g., “skin temperature is low and skin is dry”) and instructs the actions that should be taken (e.g., “set air conditioner to high temperature and activate humidifier”). Accordingly, when the user's present conditions should be improved, the condition improvement advisor 1 describes the present state to the user and instructs the user on specific actions that should be taken to improve the conditions. The advice from the condition improvement advisor 1 includes the reason for a specific action. This is effective for urging the user to comply with the advice.

After receiving the advice and reaching the house, the user sets the air conditioner at a high temperature and activates the humidifier in accordance with the advice. This increases the user's skin temperature and skin moisture content. Based on the data provided from the body temperature sensor 75 and the skin moisture sensor 95, the control circuit 36 determines that the user's skin temperature and skin moisture content are both within the normal range. The control circuit 36 activates the display 37 and the speaker 38 to notify the user that the skin temperature and skin moisture content have both been improved and returned to normal. For example, the control circuit 36 shows a text message or a picture message (e.g., icon or dynamic image) on the display 37. The content of the text message or picture message may be: “Skin temperature and skin moisture content returned to normal. Set air conditioner to lower temperature and weaken humidifier.” Further, the control circuit 36 outputs the content of the message from the speaker 38.

In this manner, in the first example, after the condition improvement advisor 1 issues advice, the control circuit 36 stops outputting that advice when determining that there is no longer a need to improve the user's conditions. Further, the control circuit 36 may output new advice in lieu of the previous advice. For example, the new advice may update the user's present state (e.g., “skin temperature and skin moisture content returned to normal”). When necessary, the new message may also include instructions to the person provided with the message (user or third person) for actions that should be taken (e.g., “set air conditioner to lower temperature and weaken humidifier”). Accordingly, after the issuance of advice, when the user's conditions no longer needs to be improved, the condition improvement advisor 1 updates the present state to the user and stops issuing the previous advice.

In a second example, the user becomes excited when driving a vehicle.

When in a state of excitement, the user's blood pressure, skin temperature, and heart beat increase. The skin temperature detected by the body temperature sensor 75 becomes greater than a reference skin temperature (enters an abnormal range), and the heart beat detected by the heart beat sensor 85 becomes greater than a reference heart beat (enters an abnormal range). From the data provided from the body temperature sensor 75 and the heart beat sensor 85, the control circuit 36 determines that the user's skin temperature is greater than the reference skin temperature and that the user's heart beat is greater than the reference heart beat.

Then, the control circuit 36 activates the display 37 and the speaker 38 and issues advice for decreasing the user's skin temperature to the reference skin temperature so that the skin temperature enters a normal range. Further, the control circuit 36 issues advice for decreasing the user's heart beat to the reference skin moisture content so that the heart beat enters a normal range. For example, the control circuit 36 shows a text message or a picture message (e.g., icon or dynamic image) on the display. The content of the text message or picture message may be: “You are excited. Lower the air conditioner temperature and relax. Listen to your favorite music.” Further, the control circuit 36 outputs the content of the message from the speaker 38.

The advice describes the user's present condition (e.g., “you are excited”) and instructs actions that should be taken (e.g., “lower the air conditioner temperature and relax. Listen to your favorite music”). Accordingly, when the user's present condition should be improved, the condition improvement advisor 1 describes the present state to the user and instructs the user on specific actions that should be taken to improve his or her condition. The advice from the condition improvement advisor 1 includes the reason for a specific action. This is effective for urging the user to comply with the advice.

After receiving the advice, the user lowers the temperature of the vehicle air conditioner and listens to his or her favorite music. This relaxes the user and lowers the user's skin temperature and heart beat. Based on the data provided from the body temperature sensor 75 and the heart beat sensor 85, the control circuit 36 determines that the user's skin temperature and heart beat are both within the normal range.

Then, the control circuit 36 the activates the display 37 and the speaker 38 to notify the user that the skin temperature and heart beat have both been improved and returned to normal. For example, the control circuit 36 shows a text message or a picture message (e.g., icon or dynamic image) on the display 37. The content of the text message or picture message may be: “You are now calm. You may stop the air conditioner and the music.” Further, the control circuit 36 outputs the content of the message from the speaker 38.

The new advice updates the user's present state (e.g., “you are now calm”) and instructs the person receiving the message (user or third person) of the actions that should be taken (e.g., “you may stop the air conditioner and the music”). Accordingly, after the issuance of advice, when the present user state no longer needs to be improved, the condition improvement advisor 1 updates the present state to the user and stops issuing the previous advice.

When the user does not comply with the advice or when the user complies with the advice but cannot improve his or her condition, the control circuit 36 continues to receive data indicating that the user is in a state of excitement. In the second example, such a state is determined when the user's skin temperature and heart beat remain high.

When the conditions are not improved, the control circuit 36 continues to issue the previous advice through the display 37 and the speaker 38. Alternatively, when a predetermined time elapses (e.g., five minutes) without condition improvements from when the previous advice is issued, the control circuit 36 issues new advice through the display 37 and the speaker 38. For example, the control circuit 36 shows a text message or a picture message (e.g., icon or dynamic image) on the display 37. The content of the text message or picture message may be “You are still excited. Stop the car and take a rest.” Further, the control circuit 36 outputs the content of the message from the speaker 38.

The new advice updates the user's present state (e.g., “you are still excited”) and instructs the person provided with the message (user) of actions that should be taken (e.g., “stop the car and take a rest”). Accordingly, when the user's present state needs to be improved, the condition improvement advisor 1 updates the present state to the user and stops issuing the previous advice. The advice from the condition improvement advisor 1 includes the reason for a specific action. This is effective for urging the user to comply with the advice.

If the same advice continues to be issued over a relatively long period of time when the conditions are not improved, this may irritate the user. To avoid such a state, the advice may be issued intermittently in predetermined cycles. Alternatively, the advice may be issued for a first time or a second time through the display 37 and the speaker 38 and issued subsequently through only the display 37.

In a third example, the user stops driving a vehicle and becomes ill when walking home such that the user cannot stand by oneself.

For example, when the user has a heatstroke outdoors on a highly humid and hot day, the control circuit 36 receives abnormal data from the ambient temperature sensor 45, the humidity sensor 55, the ultraviolet intensity or dose sensor 65, the body temperature sensor 75, the heart beat sensor 85, and the skin moisture sensor 95.

In this case, the control circuit 36 shows a text message or a picture message (e.g., icon or dynamic image) on the display 37. The content of the text message or picture message may be: “This person is having a heatstroke. Please call for an ambulance. Please give water to this person in the shade.” Further, the control circuit 36 outputs the content of the message from the speaker 38.

The advice includes the user's present state (e.g., “this person is having a heatstroke”) and instructions to the person provided with the message (user) for the actions that should be taken (e.g., “Please call for an ambulance. Please give water to this person in the shade”). Accordingly, when the user's present conditions should be improved, the condition improvement advisor 1 describes the present state to the user or the person provided with the message and gives instructions on specific actions that should be taken to improve the conditions. The advice from the condition improvement advisor 1 includes the reason for a specific action. This is effective for urging the user or the person provided with the message to comply with the advice.

Especially, when the user cannot take the actions that are necessary to improve his or her conditions, a third person may hear the audible message from the speaker 38 or look at the visual message on the display 37 and take the actions that are in accordance with the advice from the condition improvement advisor 1 (e.g., administer first aid or call for help). As a result, the user's conditions may be improved.

When a third person administers first aid in accordance with the advice and the physical state of the user starts to recover, the control circuit 36 determines from changes in the body temperature, heart beat, and skin moisture rate that the user's physical state is recovering. In this case, the condition improvement advisor 1 issues new advice for improving the user's conditions. For example, the control circuit 36 shows a text message or a picture message (e.g., icon or dynamic image) on the display 37. The content of the text message or picture message may be: “Body temperature and hydration level are returning to normal. Just in case, please take this person to a hospital.” Further, the control circuit 36 outputs the content of the message from the speaker 38.

The new advice updates the user's present state (e.g., “body temperature and hydration level are returning to normal”) and instructs the person provided with the message (user or third person) of actions that should be taken (e.g., “just in case, please take this person to a hospital”).

Accordingly, an ambulance responds to the distress call and transports the user (patient) to a hospital where the user receives treatment. The user's physical state recovers and the body temperature, heart beat, and skin moisture content return to normal levels. When determining that the user's physical state has recovered from abnormal body temperature, heart beat, and skin moisture content, the control circuit 36 issues a text message or a picture message (e.g., icon or dynamic image) on the display 37. The content of the text message or picture message may be: “Physical state has recovered. Go home carefully”. Further, the control circuit 36 outputs the content of the message from the speaker 38.

The message updates the user's present state (e.g., “physical state has recovered”) and instructs the person provided with the message (user) of actions that should be taken (e.g., “go home carefully”). The message advising the person (user) provided with the advice of the actions that should be taken ends the previous advice. In this manner, after issuance of the previous advice, when the present user state no longer needs to be improved, the condition improvement advisor 1 updates the present state to the user and ends the issuance of the previous advice.

The preferred embodiment has the advantages described below.

(1) When determining that the user's present conditions should be improved, the condition improvement advisor 1 advises of the actions that should be taken to improve the user's conditions in a manner understandable to a human (user or third person) with the display 37 and the speaker 38. The person (user or third person) provided with the advice is urged to act in accordance with the advice to improve the user's conditions.

(2) When the user's present state should be improved, the condition improvement advisor 1 specifically advises the actions that should be taken by the person provided with the advice through the display 37 and the speaker 38. Accordingly, the person receiving the advice is notified of the specific actions that should be taken to improve the user's conditions. This is effective for improving conditions.

(3) When the user's present state should be improved, the condition improvement advisor 1 issues advice describing the user's present state and the specific actions (measures) that should be taken through the display 37 and the speaker. The description of the user's present state is helpful for urging the person provided with the advice to comply with the advice. In this manner, the description of the necessity (reason) to take actions that are in accordance with the advice effectively urges the person receiving the advice to comply with the advice.

(4) After the issuance of advice, when determining that the user's present state no longer needs to be improved, the condition improvement advisor 1 stops issuing the advice, which is issued for condition improvement. This prevents repetitive issuance of unnecessary advice.

(5) After the issuance of advice, when determining that the user's present state no longer needs to be improved, the condition improvement advisor 1 issues a message updating the present state and stops issuing the previous advice. This notifies the person receiving the advice of why the issuance of the advice has been stopped and prevents that person from taking unnecessary actions.

(6) The advice includes an audible message, which is issued through the speaker 38. In addition to the user of the condition improvement advisor 1, the advice is transmitted to a third person who is near the user. Accordingly, in a case in which the user of the condition improvement advisor cannot take actions in accordance with the advice as in the above-described third example, a third person can help the user to improve the user's condition.

(7) The advice includes an audible message issued through the speaker 38 and a visual message shown on the display 37. After the speaker 38 stops issuing the audible message, the display 37 may continue to issue the visual message. Thus, in case the user cannot hear the audible message, the display 37 may be used to urge the user to take the necessary actions for improving the user's condition.

It should be apparent to those skilled in the art that the present invention may be embodied in many other specific forms without departing from the spirit or scope of the invention. Particularly, it should be understood that the present invention may be embodied in the following forms.

The display 37 and the speaker 38 do not have to be incorporated in the main unit 3. For example, the display 37 and the speaker 38 may be arranged in a vehicle or a building. Alternatively, the display 37 and the speaker 38 may be arranged in both a vehicle and a building. In this case wired or wireless communication is performed between the condition improvement advisor 1 and another independent advisor (i.e., an advisor that issues visual advice such as the display or an advisor that issues audible advice such as the speaker). The communication transmits data related with the content of advice from the condition improvement advisor to the other advisor. The other advisor that receives the data may generate advice such as those of the preferred embodiment or further detailed advice.

When arranging an independent advisor for showing a visual message (text, icon, dynamic images, etc.) or an independent advisor for issuing an audible message in a vehicle, the display or speaker for a car navigation system may be used as the independent advisors. Further, when arranging an independent advisor in a building, a personal computer or home appliance (stereo equipment or television set) may be used as the independent advisor.

Such structures eliminate the need for the display 37 and the speaker 38 from the main unit 3, which is worn by the user. This enables reduction in size and weight of the main unit 3 and provides a compact wearable main unit 3. Further, instead of incorporating the display 37 in the main unit 3, a large external display may be use to a show a large amount of detailed information in a single screen. In this case, detailed advices may be issued through the external display.

At least one of the sensor units may be incorporated in the main unit 3.

Sensor units such as an atmospheric pressure sensor unit for detecting atmospheric pressure at the location of the user, an oxygen concentration sensor unit for detecting oxygen concentration at the location of the user, a blood pressure unit for detecting the user's blood pressure, a wrinkle advancement sensor unit for detecting the advancement of the user's wrinkles (or future advancement of wrinkles), a skin fineness sensor unit for detecting fineness of the user's skin, and a skin elasticity sensor unit for detecting elasticity of the user's skin may be used.

The condition improvement advisor 1 is not limited to a wrist watch type device or bracelet type device worn on the user's arm, and may be a necklace type device or pendant type device worn around the neck of the user. Alternatively, the condition improvement advisor 1 may be an eyeglass type device or a ring type device. If the conditions monitored by the condition improvement advisor I is used as the user's physical information, it is preferred that the condition improvement advisor 1 be shaped to come into close contact with the user. If the user of the condition improvement advisor 1 is the driver of a vehicle, it is preferred that, for example, the main unit 3 be integrated with a wrist watch, which is normally worn by the user regardless of whether or not the user rides the vehicle. In this case, the sensor units desired by the user are attachable to the wrist watch.

The supporting member is not limited to the band 2, and other supporting members, such as belt, a chain, a ring, eyeglasses, a safety pin, and the like may be used.

The main unit 3 of the preferred embodiment may be used with an electronic key for a so-called keyless system that enables the locking and unlocking of a door for a vehicle, such as an automobile, or a building, such as a house, with a door lock controller when an unlock button or lock button of the electronic key is operated. In such a case, the main unit 3 incorporates functions (keyless functions) necessary for the electronic key. For example, the main unit 3 includes a function for transmitting an ID code signal including an unlock request code (lock request code) for requesting unlocking (locking) of the door and a unique ID code for the main unit 3 (electronic key) when an unlock button (or a lock button) is operated. When such an ID code signal is received by the door lock controller, the ID code is verified with a reference ID code, and the unlocking (locking) of the door is enabled when the ID codes match.

Alternatively, the main unit 3 of the preferred embodiment may be used with an electronic key for a so-called smart system that enables the locking and unlocking of a door for a vehicle, such as an automobile, or a building, such as a house, with a door lock controller when the proper key is located near the door. In such a case, the main unit 3 incorporates functions (keyless functions) necessary for the electronic key. For example, the main unit 3 includes a function for transmitting an ID code signal including a unique ID code for the main unit 3 (electronic key) in response to a request signal cyclically transmitted from a door lock controller that monitors the approach of the main unit 3 to the door. When such an ID code signal is received by the door lock controller, the ID code is verified with a reference ID code, and the unlocking (locking) of the door is enabled when the ID codes match.

If the smart function is incorporated in the main unit 3, in addition to the locking and unlocking of a door, the starting of the vehicle engine may also be enabled.

If the main unit 3 incorporates either one or both of the keyless function and the smart function, a user such as the driver of a vehicle or the resident of a building does not need to carry both of the electronic key and the condition improvement advisor 1. This would improve the portability. If a vehicle and a building both have the keyless function and the smart function, the user does not need to hold three devices, which are the vehicle key, the building key, and the condition improvement advisor 1. This would further improve the portability.

If the keyless function is incorporated in the main unit 3, the information related to the user's environmental state or physical state is transmitted to a base control device when transmitting the ID code signal from the main unit 3 to the base control device, which is arranged in the vehicle or the building. Such information may be used in the base control device.

If the smart function is incorporated in the main unit 3, the information related to the user's environmental state or physical state is transmitted to a base control device when transmitting the ID code signal from the main unit 3 to the base control device, which is arranged in the vehicle or the building. Such information can be used in the base control device. On the other hand, if the base control device transmits the information on the vehicle or the building to the main unit 3 when transmitting the request signal from the base control device to the main unit 3, such information may be used in the main unit 3. For instance, when changing the setting of the base control device, the user may be notified of the change and requested for a so-called answer back.

The present examples and embodiments are to be considered as illustrative and not restrictive, and the invention is not to be limited to the details given herein, but may be modified within the scope and equivalence of the appended claims.

1. A condition improvement advisor for monitoring a condition related to a user carrying the condition improvement advisor and improving the monitored condition, the condition improvement advisor comprising:
a sensor unit for detecting the monitored condition and outputting data representing the user's present state when carried by the user;
a present state determination circuit for determining from the data representing the user's present state whether or not the user's present state should be improved, wherein when determining that the user's present state should be improved, the present state determination circuit generates advice indicating an item that would improve the present state; and
an output device for outputting the advice generated by the present state determination circuit in a form understandable to a human.

2. The condition improvement advisor according to claim 1, wherein the advice includes a message indicating a specific action that should be taken by a person provided with the advice to improve the user's present state.

3. The condition improvement advisor according to claim 1, wherein the present state determination circuit generates a message indicating a condition that needs improvement as determined by the present state determination circuit and a specific action that should be taken by a person provided with the advice to improve the user's present state.

4. The condition improvement advisor according to claim 1, wherein after generation of the advice, when determining with the data representing the user's present state from the sensor unit that the user's present state no longer needs to be improved, the present state determination circuit stops generating the advice.

5. The condition improvement advisor according to claim 4, wherein after generation of the advice, when determining with the data representing the user's present state from the sensor unit that the user's present state no longer needs to be improved, the present state determination circuit generates a message updating the user's present state and stops generating the advice.

6. The condition improvement advisor according to claim 5, wherein the present state determination circuit generates a message indicating a new action that should be taken by a person provided with the message corresponding to the user's updated present state and output the message indicating the new action through the output device.

7. The condition improvement advisor according to claim 1, wherein:
the advice includes a visual message and an audible message; and
the output device includes a display for showing the visual message and a speaker for issuing the audible message.
